(19) United States
(12) Patent Application Publication
(10) Pub. No.: US 2016/0015621 A1
(43) Pub. Date: Jan. 21, 2016

(54) Sunscreen Formulations

(71) Applicant: AKZO NOBEL CHEMICALS INTERNATIONAL B.V., Amersfoort (NL)

(72) Inventors: Gary Theodore Martino, Monmouth Junction, NJ (US); Michael Russell, Madison, NJ (US)

(21) Appl. No.: 14/772,659

(22) PCT Filed: Mar. 10, 2014
(86) PCT No.: PCT/EP2014/054511

§ 371 Date: Sep. 3, 2015

Related U.S. Application Data
(60) Provisional application No. 61/777,579, filed on Mar. 12, 2013.

(30) Foreign Application Priority Data
Jun. 28, 2013 (EP) 13174211.6

(51) Int. Cl. A61K 8/81; A61K 8/37; A61K 8/41; A61K 8/35; A61Q 17/04; A61K 8/06

(52) U.S. Cl. CPC A61K 8/8158; A61Q 17/04; A61K 8/8152; A61K 8/8147; A61K 8/062; A61K 8/41; A61K 8/35; A61K 8/37

(57) ABSTRACT

Sunscreen formulations with improved thickening and waterproofmg properties include a polymer prepared from monomers including: styrene, a (meth)acrylic acid-containing monomer and an ester of acrylic acid or methacrylic acid or an acryl amide monomer, and an acrylic acid homopolymer thickener.

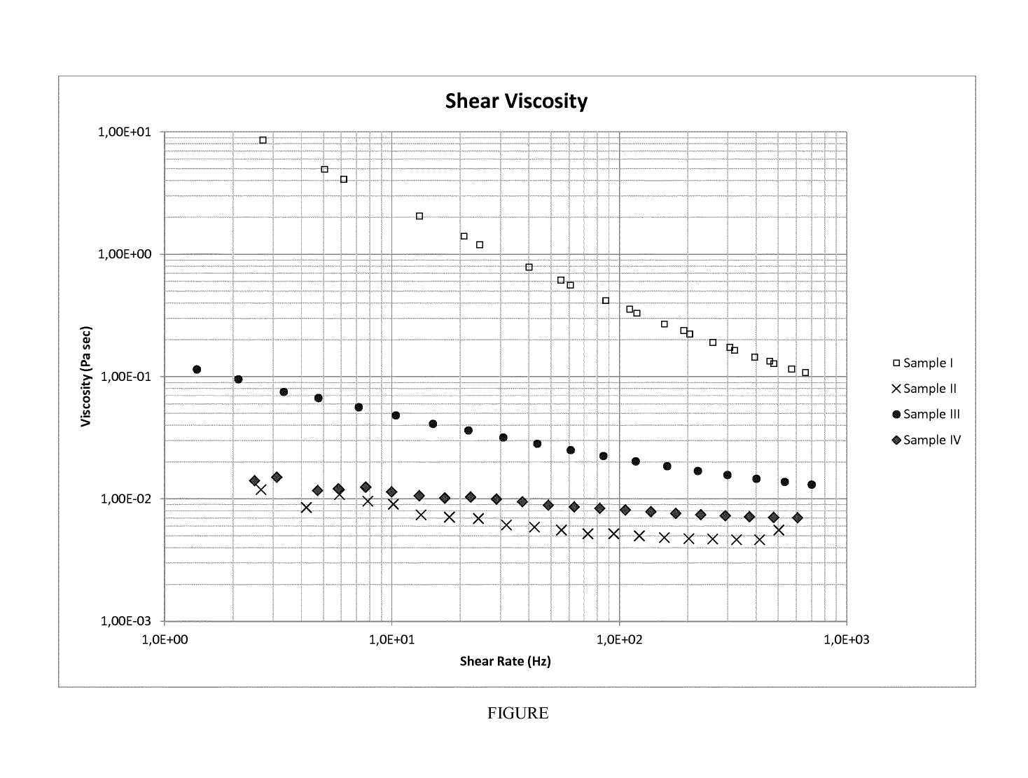

### FIELD OF THE INVENTION

The present invention relates to sunscreen formulations having improved thickening and waterproofing properties. More specifically, the present invention relates sunscreen formulations containing a styrene/acrylates polymer in combination with an acrylic acid homopolymer thickening agent.

### BACKGROUND OF THE INVENTION

Sunscreen compositions are typically categorized as either aqueous or non-aqueous compositions. Aqueous sunscreen compositions are typically creams formed as emulsions containing the active UV absorbing compounds and additional ingredients such as waterproofing agents, fragrances, emollients and other skin care ingredients. Non-aqueous sunscreen compositions are those that are typically solvent-based compositions that can be formed as gels for topical application or sprayed-on, for example from an alcohol based solution of the ingredients.

In order to keep the emulsion stable in the cream, thickeners are generally utilized. The most common thickeners are formed from cross-linked acrylic acid and are commonly known as carbomers (e.g., Carbomer 940).

The thickening properties of these cross-linked acrylic acid polymers requires that at least a portion of the acid groups are neutralized with a base, which in turn makes the polymer water swellable. Depending on the amount of neutralization and pH of the system, as well as other factors, the desired viscosity and rheology can be obtained. There is generally no interaction between the waterproofing polymers used in conventional sunscreen formulations and the polyacrylic acid. Accordingly, there is a desire for improved sunscreen formulations that can provide synergies among the various ingredients.

### SUMMARY OF THE INVENTION

This invention relates to a sunscreen formulation comprising at least one styrene/acrylates polymer (as the waterproofing polymer), at least one at least one acrylic acid homopolymer thickener and at least one sunscreen active agent. In an embodiment, the styrene/acrylates polymer is prepared from monomers comprising styrene, at least one (meth)acrylic acid-containing monomer and an ester of acrylic acid or methacrylic acid or an acrylamide monomer.

### BRIEF DESCRIPTION OF THE DRAWING

The invention is best understood from the following detailed description when read in connection with the accompanying drawing. Included in the drawing is the following FIGURE:

The FIGURE is a plot of shear viscosity of a polymer in combination with an acrylic acid homopolymer thickener according to the invention as compared to the shear viscosity of a conventional acrylates copolymer or a styrene/acrylates copolymer in combination with the same acrylic acid homopolymer thickener.

### DETAILED DESCRIPTION OF THE INVENTION

The present invention generally relates to sunscreen formulations comprising at least one waterproofing agent, at least one acrylic acid homopolymer thickener, and at least one sunscreen active agent. In an embodiment, the waterproofing agent is a styrene/acrylates polymer prepared from monomers comprising at least styrene, at least one (meth)acrylic acid-containing monomer and at least one ester of acrylic acid or methacrylic acid or an acrylamide monomer.

In an embodiment of the invention, the (meth)acrylic acid containing monomer in the polymer can range from about 0.5% to about 40% by weight of the total polymer weight (on a dry basis). In an embodiment of the invention, the (meth)acrylic acid content will range from about 2% to about 10% by weight based on total polymer weight (dry basis). In addition, in an embodiment the styrene content can range from about 25% to about 80% by weight based on the total polymer weight (dry basis). In another embodiment, the styrene content will range from about 30 to about 75% by weight, more particularly from about 45 to about 70% by weight, based on the total polymer weight (dry basis). It has surprisingly been found that when a styrene/acrylates polymer (as the waterproofing polymer) is used in conjunction with the thickening agent, such as carbomer, a significant increase in viscosity occurs as compared to waterproofing polymers that do not contain styrene or other commercially available styrene/acrylates polymers. This synergy translates to reduced cost (less thickener needed) and better skin feel in cream type sunscreen formulations.

In an embodiment, the (meth)acrylic acid monomer is chosen from acrylic acid, methacrylic acid or a combination of acrylic acid and methacrylic acid.

In addition to the styrene and (meth)acrylic acid, further monomers can include, but are not limited to, esters of acrylic acid and methacrylic acid. Some non-limiting examples of these monomers are ethyl acrylate, butyl acrylate, octyl acrylate, butyl methacrylate, ethyl methacrylate octyl methacrylate and combinations thereof.

Other monomers can also be used, such as acrylamide type monomers. These monomers may include from about 2 to about 12 carbon atoms in the alkyl group. Some non-limiting examples of suitable acrylamides include, but are not limited to, N-t-octyl acrylamide, N-butyl acrylamide, N-methyl acrylamide, methacrylamide, N-n-butyl acrylamide, N-n-octyl acrylamide, N-t-butyl acrylamide and N-t-octylacrylamide, as well as mixtures thereof. In an embodiment, the acrylamides may be N-substituted acrylamides or N-substituted methacrylamides substituted with alkyl radicals containing from 2-12 carbon atoms. In a further embodiment, the applicable acrylamides and methacrylamides include N-ethyl acrylamide, N-decyl acrylamide, N-dodecyl acrylamide and mixtures thereof, as well as the corresponding methacrylamides and mixtures thereof. Optionally, mixtures of the above-described acrylamides or methacrylamides may also be used. In an embodiment, the N-substituted acrylamide or N-substituted methacrylamide is N-tert.-octyl acrylamide. In an embodiment of the current invention, the polymer may contain a mixture of one or more hydrophobic monomers. In a preferred embodiment, the N-alkyl (meth)acrylamide monomer is N-n-octyl acrylamide or N-t-octyl acrylamide.

The polymers of this invention can contain up to about 50% by weight of these further monomers based on the total weight of the polymer. In an embodiment, the further monomers will be present from about 1% to about 50% by weight, in another embodiment from about 20% to about 45% by weight of the total weight of the polymer.

In an embodiment, the aqueous styrene/acrylates polymer is an emulsion polymer. Emulsion polymerization is a type of radical polymerization that usually starts with an emulsion incorporating water, monomer, and surfactant. The most common type of emulsion polymerization is an oil-in-water emulsion, in which droplets of monomer (the oil) are emulsified (with surfactants) in a continuous phase of water.

The waterproofing polymer may be present in the sunscreen formulation from about 0.5% to about 10% by weight based on the total sunscreen formulation weight. In an embodiment of this invention, the formulation will contain from about 1 to about 5% of the waterproofing polymer. Suitable waterproofing polymers of this invention include, for example, DERMACRYL®E, which is available from Akzo Nobel Surface Chemistry LLC, Bridgewater N.J.

The sunscreen formulation also includes from about 0.05% to about 2% by weight of the acrylic acid homopolymer thickener, more particularly from about 0.1% to about 1% by weight of the acrylic acid homopolymer thickener, and most particularly from about 0.5% to 1% by weight of the acrylic acid homopolymer thickener, based on the total formula weight. In an embodiment, the acrylic acid homopolymer thickener is carbomer. A carbomer is a family of homopolymers of acrylic acid, which is cross-linked, or bonded, with any of several polyalcohol allyl ethers.

In an embodiment, the acrylic acid-based thickening system may also have some of the acid groups neutralized with a cosmetically acceptable neutralizing agent. Suitable neutralizing agents which may be utilized in this manner include, but are not limited to, alkaline metal and alkaline metal earth hydroxides, such as sodium or potassium hydroxide, ammonia, primary, secondary and tertiary amines; alkanolamines; and, hydroxyamines, such as 2-amino-2-methyl-propanol, 2-amino-2-methyl-1,3-propanediol (AMP) and triethanol amine (TEA) and mixtures thereof

Additional film forming polymers, including either synthetic or natural polymers, can be used with the waterproofing polymer/thickener system detailed above. Non-limiting examples of these additional film forming polymers include but are not limited to: from Akzo Nobel Surface Chemistry LLC, Bridgewater N.J., AMPHOMER and AMPHOMER LV-71 polymers (octylacrylamide/acrylates/butylaminoethyl methacrylate compolymer), AMPHOMER HC polymer (acrylates/octylacrylamide copolymer) BALANCE 0/55, BALANCE CR and DERMACRYL AQF polymers (acrylates copolymer), BALANCE 47 polymer (octylacrylamide/butylaminoethyl methacrylate copolymer), RESYN 28-2930 polymer (VA/crotonates/vinyl neodecanoate copolymer), RESYN 28-1310 polymer (VA/Crotonates copolymer), FLEXAN polymers (sodium polystyrene sulfonate), DynamX polymer (polyurethane-14 (and) AMP-Acrylates copolymer), RESYN XP polymer (acrylates/octylacrylamide copolymer), STRUCTURE 2001 (acrylates/steareth-20 itaconate copolymer) and STRUCTURE 3001 (acrylates/ceteth-20 itaconate copolymer); from ISP, OMNIREZ-2000 (PVM/MA half ethyl ester copolymer), GANEX P-904 (butylated PVP), GANEX V-216 (PVP/hexadecene copolymer) GANEX V-220 (PVP/eicosene copolymer), GANEX WP-660 (tricontanyl PVP), GANTREZ A425 (butyl ester of PVM/MA copolymer), GANTREZ AN-119 PVM/MA copolymer, GANTREZ ES 225 (ethyl ester of PVM/MA copolymer), GANTREZ ES425 (butyl ester of PVM/MA copolymer), GAFFIX VC-713 (vinyl caprolactam/PVP/dimethylaminoethyl methacrylate copolymer), GAFQUAT 755 (polyquatemium-11), GAFQUAT HS-100 (polyquatemium-28) AQUAFLEX XL-30 (Polyimide-1), AQUAFLEX SF-40 (PVP/Vinylcaprolactam/DMAPA Acrylates Copolymer), AQUAFLEX FX-64 (Isobutylene/Ethylmaleimide/Hydroxyethylmaleimide Copolymer), ALLIANZ LT-120 (Acrylates/C1-2 Succinates/Hydroxyacrylates Copolymer), STYLEZE CC-10 (PVP/DMAPA Acrylates Copolymer), STYLEZE 2000 (VP/Acrylates/Lauryl Methacrylate Copolymer), STYLEZE W-20 (Polyquaternium-55), Copolymer Series (PVP/Dimethylaminoethylmethacrylate Copolymer), ADVANTAGE S and ADVANTAGE LCA (VinylcaprolactamNP/Dimethylaminoethyl Methacrylate Copolymer), ADVANTAGE PLUS (VA/Butyl Maleate/Isobomyl Acrylate Copolymer); from BASF, ULTRAHOLD STRONG (acrylic acid/ethyl acrylate/t-butyl acrylamide), LUVIMER 100P (t-butyl acrylate/ethyl acrylate/methacrylic acid), LUVIMER 36D (ethyl acrylate/t-butyl acrylate/methacrylic acid), LUVIQUAT HM-552 (polyquatemium-16), LUVIQUAT HOLD (polyquatemium-16), LUVISKOL K30 (PVP) LUVISKOL K90 (PVP), LUVISKOL VA 64 (PVPNA copolymer) LUVISKOL VA73W (PVPNA copolymer), LUVISKOL VA, LUVISET PUR (Polyurethane-1), LUVISET Clear (VP/MethacrylamideNinyl Imidazole Copolymer), LUVIFLEX SOFT (Acrylates Copolymer), ULTRAHOLD 8 (Acrylates/Acrylamide Copolymer), LUVISKOL Plus (Polyvinylcaprolactam), LUVIFLEX Silk (PEG/PPG-25/25 Dimethicone/Acrylates Copolymer); from Amerchol, AMERHOLD DR-25 (acrylic acid/methacrylic acid/acrylates/methacrylates); from Rohm&Haas, ACUDYNE 258 (acrylic acid/methacrylic acid/acrylates/methacrylates/hydroxyl ester acrylates; from Mitsubishi and distributed by Clariant, DIAFORMER Z-301, DIAFORMER Z-SM, and DIAFORMER Z-400 (methacryloyl ethyl betaine/acrylates copolymer), ACUDYNE 180 (Acrylates/Hydroxyesters Acrylates Copolymer), ACUDYNE SCP (Ethylenecarboxyamide/AMPSA/Methacrylates Copolymer), and the ACCULYN rheological modifiers; from ONDEO Nalco, FIXOMER A-30 and FIXOMER N-28 (INCI names: methacrylic acid/sodium acrylamidomethyl propane sulfonate copolymer); from Noveon, FIXATE G-100 (AMP-Acrylates/Allyl Methacrylate Copolymer), FIXATE PLUS (Polyacrylates-X), CARBOPOL Ultrez 10 (Carbomer), CARBOPOL Ultrez 20 (Acrylates/C10-30 Alkyl Acrylates Copolymer), AVALURE AC series (Acrylates Copolymer), AVALURE UR series (Po lyurethane-2, Polyurethane-4, PPG-17/IPDI/DMPA Copolymer); polyethylene glycol; water-soluble acrylics; water-soluble polyesters; polyacrylamides; polyamines; polyquaternary amines; styrene maleic anhydride (SMA) resin; polyethylene amine; and other conventional polymer that is polar solvent soluble or that can be made soluble through neutralization with the appropriate base.

It has been found that the present invention provides a significant and synergistic increase in viscosity between the waterproofing polymer and the acrylic acid homopolymer thickener in the absence of any neutralizing agent. In an embodiment of this invention, the increase in viscosity will be greater than 10 times (i.e. one order of magnitude), and in another embodiment, preferably 100 times (i.e. two orders of magnitude) when compared to the same formulation when an acrylates polymer is used in place of the waterproofing polymer of the present invention, as a shear rate of one reciprocal second (Hz or 1.0×10¹ per second).

In another embodiment, addition of a neutralizing agent to the combination of the acrylic acid homopolymer thickener and the waterproofing polymer will result in even higher viscosities. In other words, while a neutralizing agent is not required to provide a synergistic viscosifying effect between the thickener and waterproofing polymer of this invention, by adding a neutralizing agent, an even higher viscosity may be attained. Similarly, less thickening agent or less neutralizing agent need be employed to obtain the same formulation viscosity as with waterproofing polymers that do not contain styrene.

In an embodiment, the acrylic acid homopolymer thickener is neutralized from about 1% to about 100% neutralized. In another embodiment, the acrylic acid homopolymer thickener is 5% to 75% neutralized.

For purposes of the present invention, a “sunscreen active agent” or “sunscreen active” shall include all of those materials, singly or in combination, that are regarded as acceptable for use as active sunscreening ingredients based on their ability to absorb UV radiation. Such compounds are generally described as being UV-A, UV-B, or UV-A/UV-B active agents. Approval by a regulatory agency is generally required for inclusion of active agents in formulations intended for human use. Those active agents which have been or are currently approved for sunscreen use in the United States include organic and inorganic substances including, without limitation, para aminobenzoic acid, avobenzone, cinoxate, dioxybenzone, homosalate, menthyl anthranilate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum. Examples of additional sunscreen actives that have not yet been approved in the US but are allowed in formulations sold outside of the US include ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate and mixtures thereof However, as the list of approved sunscreens is currently expanding, those of ordinary skill will recognize that the invention is not limited to sunscreen active agents currently approved for human use but it is readily applicable to those that may be allowed in the future.

In an embodiment of the invention, the sunscreen active agents are present in an amount of about 50wt % or less by weight of the formulation, preferably about 40wt % or less, and more preferably about 30wt % or less by weight of the formulation, and in still yet more preferably about 25wt % or less by weight of the formulation. In an embodiment, the sunscreen active agents may be included from about 0.5 wt % or more, more preferably from 1.0 wt % or more, and still more preferably from 5 wt % or more based on the weight of the formulation

In one embodiment of the invention, the sunscreen active agent comprises a photoprotecting effective amount of particulates of at least one inorganic pigment or nanopigment, non-limiting examples of which include titanium dioxide, zinc oxide, iron oxide, zirconium oxide, cerium oxide, or mixture thereof.

In an embodiment, the sunscreen formulation is in the form of an oil-in-water emulsion. It is also within the scope of the present invention that formulations where the sunscreen active agent is an ingredient in another topically applied composition are also included. Some non-limiting examples are lipstick, make-up, lip-balm, eye-shadow, hair dyes and conditioners, or any application where sun protection may be deemed beneficial.

The formulations of the present invention may contain a wide range of additional, optional components, which are referred to herein as “cosmetic components”, but which can also include components generally known as pharmaceutically active agents. The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions of the present invention. Examples of these functional classes disclosed in this reference include: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, SPF boosters, waterproofing agents, and viscosity increasing agents (aqueous and nonaqueous).

Unless otherwise specified, all the percentages listed above are based on the total weight of the formulation. One of skill in the art would recognize that some ingredients are not provided as dry materials, so the percentages of those ingredient are meant as the dry solids as a percent of the total formulation. Alternatively stated, the water or solvent in an ingredient is not taken into account for purposes of calculating the percentage in the formulation.

The following examples are intended to exemplify the present invention but are not intended to limit the scope of the invention in any way. The breadth and scope of the invention are to be limited solely by the claims appended hereto.

### EXAMPLES

### Rheological Measurements

Rheological properties of samples were run using a Rheometric Scientific SR-5000 stress rheometer. The geometry of the attachment for a measurement was a 40 mm-parallel plate with the gap between the plates being 0.5 mm. Two rheological properties were evaluated, viscoelasticity and shear viscosity hysteresis. For viscoelasticity, the percentage of strain during the measurement was set at 5%, and the frequency range was evaluated between 0.1 and 100 radius/sec. The elastic modulus, viscous modulus, and tans during these measurements were recorded. For the shear viscosity hysteresis, the rheometer was set with incremental shear stress increase starting from the minimum stress limit until the shear rate reaches the order close to 1000/sec, then shear stress was set to be incrementally reduced to the minimum stress the instrument can detect. The shear viscosity was recorded during this process.

### Materials

The following formulations were prepared with DERMACRYL® E (styrene/acrylates copolymer) and DERMACRYL® AQF (acrylates copolymer) as the two waterproofing agents (available from Akzo Nobel Surface Chemistry LLC, Bridgewater N.J..) The formulations were evaluated for sunscreen protection factor (SPF), both static and following 80 minutes of water immersion, using the following procedure (courtesy of: Florida Suncare testing Co., Ormand Beach, Fla.).

### Protocol for the Evaluation of Sun Protection Factor (SPF) Static and Following 80 Minutes Water Immersion

### I. Objective

To measure the Sun Protection Factor (SPF) value for three sunscreen formulas, Static and following 80 minutes water immersion, using FDA, 21 CFR Sec. 201.327, subpart (i), SPF Test Procedure, Sunscreen Drug Products for Over-the-Counter Human Use, Final Monograph, Federal Register, Vol. 76, No. 117, Jun. 17, 2011.

### II. Study Type

Five (5) subject Screener, Static and 80 Minute Water Resistant SPF, with a final report furnished to the sponsor, which includes the mean SPF values and projected Label SPF values for the two test sunscreen products submitted by the sponsor.

### III. Experimental Design

A. Selection of Test Subjects

Fair-skinned subjects, male and female, eighteen years of age or older, of skin types I, II, or III as defined in FDA, 21 CFR, Sec. 201.327, subpart (i), SPF Test Procedure, (3) Test Subjects, (ii) Medical History, (B) Skin Type, Jun. 17, 2011.

a. Inclusion Criteria
1. Individuals eighteen years of age or older.
   2. Individuals with fair, uniformly-colored skin on the lower area of the back which would allow a discernable erythema.
   3. Individuals free of any dermatological or systemic disorder which, in the opinion of the testing personnel, would interfere with the results of the study.
   4. Individuals in good health who have completed a preliminary medical history.
   5. Individuals who have read, understood and signed a consent document in compliance with 21 CFR 50.

b. Exclusion Criteria
1. Individuals with any visible skin disease at the study site, which in the opinion of the investigative personnel would, interfere with the study results.
2. Individuals taking medications which might affect study results, e.g. photosensitizers, antihistimines, analgesics or anti-inflammatory drugs.
3. Females who are pregnant, planning a pregnancy or nursing a child
4. Individuals with a history of skin cancer.
5. Individuals with a history of hepatitis or other blood disease.
6. Individuals with a known sensitivity to cosmetics, skin care products or topical drugs as related to product(s) being evaluated.
7. Individuals with recent sun exposure on the areas to be tested.

B. Test Materials
1. A sufficient quantity (approximately 2 oz.) of each test product should be submitted by the test sponsor to satisfy the requirements for a test panel of up to ten subjects. The test sample will be entered into a SPF testing log and assigned a unique test product sample identification number.

C. Light Source
A Xenon Arc Solar Simulator lamp, which provides a continuous emission spectrum from 290 to 400 nanometers (nm) with a limit of 1,500 watts per square meter (W/m²) of total irradiance for all wavelengths between 250 and 1,400 nm. The spectral output of the solar simulator will be filtered so that it meets the spectral output requirements for testing Sunscreen Drug Products for over-the-counter human use; FDA Final Monograph, 21 CFR Part 201.327 (i)(1) UV Source, Federal Register, Vol. 76, No. 117, Jun. 17, 2011 and the International Sun Protection Factor (SPF) Test Method, May 2006.

D. Study Group Size
Five (5) subjects will be enrolled to obtain the required five (5) Static and 80 Minute Water Resistant (SPF) Sun Protection Factor test results for each test product in this study. Subjects deemed as technical failures on the product protected test sites will NOT be replaced during the five (5) subject “screener”. They would be replaced in a full study, at a “pro-rated” cost, if the sponsor agrees to increase the size of the test panel.

### IV. Study Procedure

Day 1

Subject Enrollment
The test subjects will report to the testing laboratory and receive a complete explanation of the study procedures. Those who wish to participate will sign a written, witness consent form, and a permission to release personal health information form and provide a brief medical history. The technician will do a final examination of the subject's back, between the belt-line and shoulder blades to determine their suitability to participate in the study.

MEDu UV Dose Administration
1. A series of 5 UV radiation doses expressed as Joules/square meter, increasing in 25% increments, will be administered to an unprotected location on the test subject's back, just below the shoulder blades and above the belt-line, to determine the initial unprotected MED (MEDu).
2. The test subjects will be instructed to avoid additional UV exposure, and to avoid taking any photosensitizing medications until the conclusion of the study.
3. The MEDu shall be administered in the following 5 dose series, with X representing the amount of UV energy projected to produce the test subject's MEDu.

| Dose 1 | Dose 2 | Dose 3 | Dose 4 | Dose 5 |
|---|---|---|---|---|
| 0.64X | 0.80X | 1.00X | 1.25X | 1.56X |

Day 2

MEDu Determination
1. Subject's will return to the testing laboratory within 16 to 24 hours following completion of the MEDu doses for evaluation of the responses, and to determine each subjects unprotected MED (MEDu).
2. The subject's Minimal Erythemal Dose (MED) is the quantity of erythema effective energy, or dose corresponding to the first site that produced the first unambiguous erythema reaction with clearly defined borders. Table 1 below shows the grading scale to be used for determining a MED (+) response.

TABLE 1

| | |
|---|---|
| − | No perceptible erythemal response |
| ? | Barely perceptible erythemal response |
| + | Unambiguous erythema reaction with clearly defined borders |
| | (MED) |
| ++ | Moderate erythema with sharp borders |
| +++ | Dark red erythema with sharp borders |

Application of Product for SPF Determination

Sunscreen application as specified in 21 CFR 201.327 (4)(ii)(iii)(iv) to (10 cm×5 cm), 50 square centimeter randomly located test sites, drawn in the designated locations on the subject's back, (between the beltline and the shoulder blade) using a template and an indelible marker. Test subsites will be a minimum of 0.5 cm² and the distance between subsites a minimum of 0.8 cm. The technician will then apply the test formulas in the test areas. The sunscreens will be applied by “spotting” the product across the test area and gently spreading, using a non-saturated finger cot compatible with the test product, until a uniform film is applied to the entire test area. A product density of 2 mg/cm² must be delivered to the test area. To accomplish this, the technician will weigh an amount in excess of 100 mg, to allow for the residual amount left on the finger cot (approximately 10%). The test products will be permitted to dry a minimum of 15 minutes prior to UV exposure or water resistance testing.

80 Minute Water Immersion Sequence
1. An indoor fresh water Jacuzzi maintained at 23 to 32 deg. Celsius will be used in this testing procedure. Fresh water is clean drinking water that meets the standards in 40 CFR part 141.
2. The pool and air temperature, as well as the relative humidity will be recorded prior to testing.
3. The label SPF value for the test product submitted by the sponsor will be determined after 80 minutes of water immersion using the following procedure:
   a. Apply sunscreen product. (Followed by a minimum 15 minute waiting period after application).
   b. Twenty minutes of moderate activity in the water.
   c. Fifteen minute rest period. (Do not towel test sites)
   d. Repeat steps b. and c. until a total of 80 minutes of water immersion is achieved.
   e. Conclude water test. (Allow test sites to dry completely without toweling)
4. Begin light source exposures to the 80 minute water resistant test site areas in accordance with FDA, 21 CFR, Part 201.327 (5)(iii).

MEDp UV Dose Administration
The technician will administer a series of 5 UV radiation doses expressed as Joules/square meter, as specified in 21 CFR 201.327 (5)(iii), progressively increasing in increments of 15 or 20 percent, determined by the expected SPF range of the test products. The MEDp will be administered in the following 5 dose series with X representing the expected amount of UV energy required to produce a MEDp.

Expected SPF 15 (FDA Control Static)

| Dose 1 | Dose 2 | Dose 3 | Dose 4 | Dose 5 |
|---|---|---|---|---|
| 0.69X | 0.83X | 1.00X | 1.20X | 1.44X |

Expected SPF 50 (Test Products Static and 80 Min WR)

| Dose 1 | Dose 2 | Dose 3 | Dose 4 | Dose 5 |
|---|---|---|---|---|
| 0.76X | 0.87X | 1.00X | 1.15X | 1.32X |

MEDu Repeat UV Dose Determination
On Day 2, the technician will administer a timed series of 5 UV doses, increasing in 25% increments to an unprotected location on the subject's back to determine the subject's second day MEDu. The series of 5 doses will include the original MEDu in the center as follows:

Multiple of Original MEDu (X)

| Dose 1 | Dose 2 | Dose 3 | Dose 4 | Dose 5 |
|---|---|---|---|---|
| 0.64X | 0.80X | 1.00X | 1.25X | 1.56X |

Day 3

Evaluation of Responses to UV Doses for Static and 80 Min WR SPF and Repeat MEDu
Subjects will return 16 to 24 hours following completion of the UV doses from day 2. The MED for all sites that received UV doses, both protected and unprotected areas will be evaluated and recorded. Erythema responses will be evaluated in an area that provides at least 450 lux of illumination at the test site.

### Sample Preparation:

### Sample 1

| Phase A | |
|---|---|
| Deionized Water | 95.10% |
| Carbopol 980 | 0.50% |
| Phase B | |
| Styrene/Acrylates Copolymer (45% active) | 4.40% |
| Total: | 100.00% |

### Process:

Sprinkle CARBOPOL® 980 into DI Water while mixing on a Caframo overhead mixer with a propeller blade at 600 rpm in a beaker. Mix until CAROBOPOL® 980 is fully dispersed. Add Styrene/Acrylates Copolymer to batch and mix until uniform.

### Sample 2 (Comparative)

| Phase A | |
|---|---|
| Deionized Water | 95.10% |
| Carbopol 980 | 0.50% |
| Phase B | |
| Acrylates Copolymer(45% active) | 4.40% |
| Total: | 100.00% |

### Process:

Sprinkle Carbopol 980 into DI Water while mixing on a Caframo overhead mixer with a propeller blade at 600 rpm in a beaker. Mix until CARBOPOL® 980 is fully dispersed. Add Acrylates Copolymer to batch and mix until uniform.

Sample 3—SPF50 Sunscreen emulsion

| Phase A | |
|---|---|
| Deionized Water | 53.40% |
| Dissolvine ® NA2-S | 0.10% |
| Phenonip | 1.00% |
| Carbopol Ultrez 21 | 0.20% |
| Triethanolamine-99% | 0.20% |
| Styrene/Acrylates Copolymer(45% active) | 4.40% |
| Phase B | |
| Avobenzone | 3.00% |
| Homosalate | 13.00% |
| Ethylhexyl Salicylate | 5.00% |
| Octocrylene | 5.00% |
| Benzophenone-3 | 6.00% |
| C12-15 Alkyl Benzoate | 5.00% |
| Arlacel 165 | 3.70% |
| Total: | 100.00% |

Add DI Water with Disodium EDTA and Phenonip to main beaker and begin heating to 75-80° C. with low mixing(2000 rpm) on Silverson homogenizer.
Once dispersed, sift in Ultrez 21 slowly and mix at 2500 rpm until fully dispersed/dissolved (avoid clumping while adding).
Add TEA to batch and mix until fully dispersed at 2500 rpm.
Make sure you can see the gel “pulling” through the homogenizer head (adjust speed up to 3200 rpm).
Check pH of batch after neutralization with TEA.
Add Styrene/Acrylates Copolymer and homogenize until fully dispersed. Again, make sure you can see the gel “pulling” through the homogenizer head (3200 rpm).
Add Phase B ingredients to side beaker and begin heating to 75-80 C with Caframo overhead propeller mixing at speed 300-400.
Once Phase B is melted and temperature reaches 75-80° C., add Phase B to Phase A with high to moderate (3500 rpm) homogenization mixing for 10 minutes at 75-80° C.
Begin cooling batch to 45° C. (2500 rpm).
At 45° C., switch batch to sweep mixing blade on Caframo overhead mixer and mix at speed 20 until batch reaches 25° C.
An In-vivo 5 subject SPF test was run on an expected SPF 50 sunscreen emulsion(see below) for static and 80 minute water immersion containing 2% active level of Styrene/Acrylates Copolymer (56.5% styrene,
41% 2-ethylhexyl acrylate, and 2.5% acrylic acid) to show film forming and water proofing performance.

TABLE 2
Static and 80 Min WR SPF Testing
Summary
Sample Description: SPF 50 Lotion
Subjects Tested: 5

| | | MEDu | | 1901A | | 80 min |
|---|---|---|---|---|---|---|
| Subject | | Joules/M2 | Skin | FDA Std. | Static | WR |
| ID Number | Sex | Day 1/Day 2 | Type | Static SPF | SPF | SPF |
| 1313 | F | 49.58/49.58 | II | 15.00 | 66.13 | 66.13 |
| 1314 | F | 49.58/49.58 | II | 18.00 | 66.13 | 66.13 |
| 1524 | M | 49.58/49.58 | I | 15.00 | 57.50 | 57.50 |
| 1100 | M | 49.58/49.58 | II | 18.00 | 57.50 | 57.50 |
| 1523 | F | 49.58/49.58 | II | 15.00 | 57.50 | 57.50 |

| | | | |
|---|---|---|---|
| AVERAGE: | 16.20 | 60.95 | 60.95 |
| STD DEVIATION: | 1.47 | 4.23 | 4.23 |
| STD ERROR OF MEAN: | 0.33 | 0.95 | 0.95 |
| STD % ERROR OF MEAN: | 2.03 | 1.55 | 1.55 |
| A = ts/sq root n | 0.57 | 1.64 | 1.64 |
| X − A | 15.63 | 59.32 | 59.32 |
| LABEL SPF | 15 | 59 | 59 |

Based on the In-Vivo test results above, the SPF50 sunscreen emulsion containing 2% active level of Styrene/Acrylates Copolymer showed excellent film forming and water proofing performance.

The substantivity or rub-off resistance was tested using cast films of the Styrene/Acrylates Copolymer vs. another sunscreen emulsion film former Acrylates Copolymer following the procedure below:

Screening Method for Rub-off Resistance
Equipment: Glass plate (10×6 inches wide)
20 mil draw down bird applicator
Balance with weight sensitivity of 0.01
675 gram weight
Rubber gloves powder free
* Control Temperature Humidity Room (CTH Room)
Procedure: 1. Clean and dry the glass plates in a CTH room 2 hours before testing.
2. Handle all equipment with gloves to prevent the addition of moisture.
3. Weight the glass plate.
4. Draw down 20 mil (1 mil=1/1000 inch) wet film on the glass plate. A gram of polymer is placed on the inside of the applicator. The applicator is then drawn down over a glass plate producing a layer of uniform thickness of film.
5. Allow to dry for 3 hours.
6. Take weight of glass plate.
7. Take the weight fixture (675 g) and wrap it with paper towel
8. Wipe the glass plate with the weight fixture under closely controlled conditions. Only 1 pass is required.
9. Re-weight glass plate.
10. Repeat procedure (1-9) on each product in triplicates

### Calculation:

Weight of glass plate with sample−weight of glass plate=Initial Sample Weight
Weight of glass plate with sample after wipe−weight of glass plate=Weight Sample after Wipe
Weight Sample after Wipe×100=% Substantivity

### Initial Sample Weight

An average is taken of the triplicates glass plates samples and reported as % Substantivity for each sample evaluated.
* Under normal average conditions of 72-75° F. and 35-55% R.H.
(dry films in the CTH Room if conditions warrant)

1 gram of polymer was used to cast the film

| | | Weight of the | | |
|---|---|---|---|---|
| ID sample | | slide + | Weight of the | |
| (2% solution) | | sample | slide + sample | |
| 4.4 grams/95.6 of | Weight of the | (after 4 hr | after wipe | % |
| water | slide (empty) | drying) | (1 pass) | Sustantivity |
| A. Styrene/Acrylates | 539.56 | 539.58 | 539.58 | |
| Copolymer | | | | |
| B. Styrene/Acrylates | 547.26 | 547.28 | 547.28 | 100% |
| Copolymer | | | | |
| C. Styrene/Acrylates | 546.94 | 546.98 | 546.98 | |
| Copolymer | | | | |
| | Initial Sample | Wiped slide | | |
| A. Styrene/Acrylates | 0.02 | 0.02 | 1 × 100 | |
| Copolymer | | | | |
| B. Styrene/Acrylates | 0.02 | 0.02 | 1 | |
| Copolymer | | | | |
| C. Styrene/Acrylates | 0.04 | 0.04 | 1 | |
| Copolymer | | | | |

1 gram of polymer was used to cast the film

| | | Weight of the | | |
|---|---|---|---|---|
| ID sample | | slide + | Weight of the | |
| (2% solution) | | sample | slide + sample | |
| 4.4 grams/95.6 of | Weight of the | (after 4 hr | after wipe | % |
| water | slide (empty) | drying) | (1 pass) | Sustantivity |
| Acrylates | 0.04 | 0.03 | 0.75 × 100 = 75.0 | |
| Copolymer | | | | |
| Acrylates | 0.03 | 0.02 | 0.67 | 72.3 |
| Copolymer | | | | |
| Acrylates | 0.04 | 0.03 | 0.75 | |
| Copolymer | | | | |
| | Initial Sample | Wiped slide | | |
| Acrylates | 0.04 | 0.03 | 0.75 | |
| Copolymer | | | | |
| Acrylates | 0.03 | 0.02 | 0.67 | |
| Copolymer | | | | |
| Acrylates | 0.04 | 0.03 | 0.75 | |
| Copolymer | | | | |

The films cast with Styrene/Acrylates Copolymer showed superior substantivity when compared to the films cast with Acrylates Copolymer.

Certain Styrene/Acrylates Copolymers also showed the ability to thicken sunscreen formulas containing Carbopol when compared to Acrylates Copolymer and other Styrene Acrylates Copolymers, as shown the following Shear Viscosity Determination example and as illustrated in the rheological graphs in the FIGURE. This synergy afforded by the polymers of the present invention is valuable because it demonstrates that the polymers are multifunctional polymers that thus allow a reduction in the amount of thickener needed, thereby reducing the overall cost of the sunscreen.

### Shear Viscosity Determination

### Sample Preparation

Samples as shown in Table 3 were prepared as follows. Phase A was prepared by sprinkling CARBOPOL® 980 into DI Water while mixing on a Caframo overhead mixer with a propeller blade at 600 rpm in a beaker. Mix until CAROBOPOL® 980 is fully dispersed. The copolymers of Phase B were added to the batch and mixed until uniform.

TABLE 3

| | | |
|---|---|---|
| Trade Name/INCI Name | % w/w | Supplier |

Sample I

| | | |
|---|---|---|
| Phase A | | |
| Deionized Water Water (Aqua) | 95.10% | Local |
| Carbopol 980 Carbomer | 0.50% | Lubrizol Advanced |
| Phase B | | |
| DERMACRYL ® E | 4.40% | AkzoNobel Personal Care |
| (45% active) polymer | | |
| Styrene/Acrylates Copolymer | | |
| Total: | 100.00% | |

Sample II (Comparative)

| | | |
|---|---|---|
| Phase A | | |
| Deionized Water Water (Aqua) | 94.10% | Local |
| Carbopol 980 Carbomer | 0.50% | Lubrizol Advanced |
| Phase B | | |
| DERMACRYL ® AQF | 4.40% | AkzoNobel Personal Care |
| (45% active) polymer | | |
| Acrylates Copolymer | | |
| Total: | 100.00% | |
| Phase A | | |
| Deionized Water Water (Aqua) | 94.50% | Local |
| Carbopol 980 Carbomer | 0.50% | Lubrizol Advanced |
| Materials | | |
| Phase B | | |
| ACUSOL ® OP302P | 5.00% | The Dow Chemical |
| (40% active) polymer | | Company |
| Styrene/Acrylates Copolymer | | |
| Total: | 100.00% | |

Sample IV (Comparative)

| | | |
|---|---|---|
| Phase A | | |
| Deionized Water Water (Aqua) | 93.79% | Local |
| Carbopol 980 Carbomer | 0.50% | Lubrizol Advanced |
| Phase B | | |
| SYNSTRAN ® 5903 | 5.71% | Interpolymer Corporation |
| (35% active) polymer | | |
| Styrene/Acrylates Copolymer | | |
| Total: | 100.00% | |

The FIGURE shows the unique thickening synergy seen when the inventive aqueous Styrene/Acrylates Copolymers are used in combination with Carbopol®, a type of carbomer, while the Acrylates Copolymer and other commercially available Styrene/Acrylates Copolymers do not have the same effect.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described herein, the invention is not intended to be limited to the details shown. Rather, various modifications may be made in the details within the range and scope of equivalents of the claims and without departing from the spirit and scope of the invention.

1. A sunscreen formulation comprising:
at least one aqueous styrene/acrylates polymer comprising the polymerization product of styrene, at least one (meth)acrylic acid-containing monomer and an ester of acrylic acid or methacrylic acid or an acrylamide monomer;
at least one acrylic acid homopolymer thickener; and
at least one sunscreen active agent.

2. The sunscreen formulation of claim 1 wherein the sunscreen formulation is in the form of an oil-in-water emulsion.

3. The sunscreen formulation of claim 1 wherein the (meth)acrylic acid-containing monomer is acrylic acid.

4. The sunscreen formulation of claim 1 wherein the (meth)acrylic acid-containing monomer is present in the polymer from about 0.5 weight percent to about 40 weight percent, preferably from about 2.0 weight percent to about 10 weight percent, of the total polymer weight.

5. The sunscreen formulation of claim 1 wherein the styrene monomer is present in the styrene/acrylates polymer from about 25 weight percent to about 80 weight percent of the total styrene/acrylates polymer weight.

6. The sunscreen formulation of claim 1 wherein the acrylamide monomer is chosen from N-t-octyl acrylamide, N-butyl acrylamide, N-methyl acrylamide, methacrylamide, N-n-butyl acrylamide, N-n-octyl acrylamide, N-t-butyl acrylamide, N-t-octylacrylamide or mixtures thereof.

7. The sunscreen formulation of claim 1 wherein the acrylamide monomer is chosen from N-substituted acrylamides or N-substituted methacrylamides substituted with alkyl radicals containing from 2-12 carbon atoms.

8. The sunscreen formulation of claim 1 wherein the at least one aqueous styrene/acrylates polymer is an emulsion polymer.

9. The sunscreen formulation of claim 1 wherein the acrylic acid homopolymer thickener is present in formulation from about 0.05 weight percent to about 2 weight percent of the total formulation weight.

10. The sunscreen formulation of claim 1 wherein the styrene/acrylates polymer is present in the formulation from about 0.5 weight percent to about 10 weight percent of the total formulation weight.

11. The sunscreen formulation of claim 1 further comprising a neutralizing agent.

12. The sunscreen formulation of claim 1 wherein the neutralizing agent is selected from the group consisting of alkaline metal and alkaline metal earth hydroxides, ammonia, primary, secondary amines, tertiary amines, alkanolamines hydroxyamines and mixtures thereof.

13. The sunscreen formulation of claim 1 wherein the at least one acrylic acid homopolymer thickener is neutralized from about 1% to about 100%.

14. The sunscreen formulation of claim 1 wherein the at least one sunscreen active agent is present from 0.5 weight percent to about 50 weight percent based on total weight of the formulation.

15. The sunscreen formulation of claim 1 wherein the at least one sunscreen active agent is selected from the group consisting of para aminobenzoic acid, avobenzone, cinoxate, dioxybenzone, homosalate, menthyl anthranilate, octyl salicylate, oxybenzone, padimate O, phenylbenzimidazole sulfonic acid, sulisobenzone, trolamine [symbol]
